(19) United States
(12) Patent Application Publication
(10) Pub. No.: US 2025/0331915 A1
(43) Pub. Date: Oct. 30, 2025

(54) MEDICAL SYSTEMS, DEVICES, AND RELATED METHODS

(71) Applicants: Boston Scientific Scimed, Inc., Maple Grove, MN (US); Boston Scientific Medical Device Limited, Galway (IE)

(72) Inventors: Takahiro KOBAYASHI, Tokyo (JP); Kensuke HAYASHI, Penang (MY); Juan Pablo ORTIZ GARCIA, Heredia (CR); Katherin NARVAEZ NUÑEZ, Cartago (CR)

(73) Assignees: Boston Scientific Scimed, Inc., Maple Grove, MN (US); Boston Scientific Medical Device Limited, Galway (IE)

(21) Appl. No.: 19/185,875

(22) Filed: Apr. 22, 2025

Related U.S. Application Data
(60) Provisional application No. 63/638,605, filed on Apr. 25, 2024.

(51) Int. Cl. A61B 18/14; A61B 18/00

(52) U.S. Cl. CPC A61B 18/1482; A61B 2018/00535; A61B 2018/00577; A61B 2018/00601; A61B 2018/00982; A61B 2018/144; A61B 2018/1465; A61B 2218/002; A61B 2218/007

(57) ABSTRACT

A medical device may comprise: a handle including an actuator; a shaft extending from the handle, wherein the shaft includes a wire lumen extending longitudinally through the shaft; and a wire positioned within the wire lumen and extending from the handle to a distal end of the shaft. A distal portion of the wire extends (i) out of a rear-side opening of the shaft and (ii) into a tip-side opening of the shaft such that an exposed portion of the wire is outside of the shaft between the rear-side opening and the tip-side opening; and a distal end of the wire is coupled to the shaft.

### CROSS-REFERENCE TO RELATED APPLICATIONS

This application claims the benefit of priority of U.S. Provisional Patent Application No. 63/638,605, filed on Apr. 25, 2024, the entirety of which is incorporated herein by reference.

### TECHNICAL FIELD

Various aspects of this disclosure generally relate to medical systems, devices, and methods for manipulating or treating tissue or other material within a body. In particular, aspects of the disclosure relate to medical systems, devices, and methods for performing a medical procedure using a medical device capable of being inserted through an insertion device and into the body to cut or ablate a target site and deflecting or otherwise positioning the medical device relative to a distal end of the insertion device.

### BACKGROUND

A wide variety of medical techniques and instruments have been developed for diagnosis and/or treatment within a patient's body, such as within a patient's gastrointestinal (GI) tract. Endoscopic sphincterotomy (EST), endoscopic retrograde cholangiopancreatography (ERCP), endoscopic sub-mucosal dissection (ESD), endoscopic sub-mucosal resection (ESR), mucosal resection (EMR), polypectomy, mucosectomy, etc., are minimally invasive treatment and/or diagnostic methods or portions such methods for both malignant and non-malignant lesions. Endoscopic medical procedures, such as, for example, EST, may be used to incise tissue (e.g., the papillary bile duct) to allow access to a body lumen. Often, cutting angle for an incision is important to mitigate patient risk and limit hemorrhage and perforation in EST procedures. However, many conventional sphincterotomes or other medical devices for cutting tissue have limited ability to adjust the cutting angle, and deflection of the sphincterotome (or another end effector) is often limited and/or dependent on the tip deflection of an endoscope or other device used for insertion into the patient. Furthermore, devices with fixed cutting instruments (e.g. knife angle, etc.) may be difficult to maneuver without causing injury to the patient, may cause an increased cognitive load on the operator(s) to obtain the desired cutting position, may increase procedure time, and/or create other issues. These concerns may increase the duration, costs, and risks of the medical procedure. The devices and methods of this disclosure may rectify some of the deficiencies described above or address other aspects of the art.

### SUMMARY

Examples of this disclosure relate to, among other things, systems, devices, and methods for performing one or more medical procedures. Each of the examples disclosed herein may include one or more of the features described in connection with any of the other disclosed examples.

In one example, a medical device may comprise: a handle including an actuator; a shaft extending from the handle, wherein the shaft includes a wire lumen extending longitudinally through the shaft; and a wire positioned within the wire lumen and extending from the handle to a distal end of the shaft. A distal portion of the wire extends (i) out of a rear-side opening of the shaft and (ii) into a tip-side opening of the shaft such that an exposed portion of the wire is outside of the shaft between the rear-side opening and the tip-side opening; and a distal end of the wire is coupled to the shaft.

In other aspects, the medical device may include one or more of the following features. The rear-side opening may be longitudinally aligned with the tip-side opening. The rear-side opening is at least partially formed by a first pair of longitudinally-extending side walls, each side wall of the first pair of side walls extends from the wire lumen to a radially-outermost surface of the shaft, and each side wall of the first pair of side walls are substantially parallel to each other. The tip-side opening is at least partially formed by a second pair of longitudinally-extending side walls, each side wall of the second pair of side walls extends from the wire lumen to a radially-outermost surface of the shaft, each side wall of the second pair of side walls are substantially parallel to each other, and the second pair of side walls are transverse from the first pair of side walls. The tip-side opening is at least partially formed by a second pair of longitudinally-extending side walls, each side wall of the second pair of side walls extends from the wire lumen to a radially-outermost surface of the shaft, each side wall of the second pair of side walls is substantially parallel to each other, and the second pair of side walls are at least one of: (i) substantially perpendicular to the first pair of side walls, or (ii) are angled approximately forty-five degrees relative to the first pair of side walls.

In other aspects, the medical device may include one or more of the following features. The tip-side opening is at least partially formed by a first pair of longitudinally-extending side walls, each side wall of the first pair of side walls extends from the wire lumen to a radially-outermost surface of the shaft, and each side wall of the first pair of side walls is substantially longitudinally aligned with each other. The rear-side opening is at least partially formed by a second pair of longitudinally-extending side walls, each side wall of the second pair of side walls extends from the wire lumen to a radially-outermost surface of the shaft, each side wall of the second pair of side walls is substantially longitudinally aligned with each other, and the second pair of side walls are transverse to the first pair of side walls. The rear-side opening is at least partially formed by a first pair of longitudinally-extending side walls, each side wall of the first pair of side walls extends from the wire lumen to a radially-outermost surface of the shaft, and a first side wall of the first pair of side walls extends substantially perpendicular to a second side wall of the first pair of side walls. The tip-side opening is at least partially formed by a second pair of longitudinally-extending side walls, each side wall of the second pair of side walls extends from the wire lumen to a radially-outermost surface of the shaft, a third side wall of the second pair of side walls extends substantially perpendicular to a fourth side wall of the second pair of side walls, the first side wall is angled relative to the third side wall and the second side wall is angled relative to the fourth side wall.

In other aspects, the medical device may include one or more of the following features. The tip-side opening is circumferentially offset from the rear-side opening. The wire is configured to receive electrical energy. The shaft further includes a second lumen configured to receive a guidewire. The shaft further includes a third lumen configured to receive fluid. The wire, when in an actuated configuration, is configured to include both a convex portion and a concave portion relative to the shaft. The wire, when in an actuated configuration, is configured to include a peak portion, and the peak portion is closer to the tip-side opening than the rear-side opening when the wire is in an actuated state.

In other aspects, a medical device may comprise: a shaft extending distally to a distal end, wherein the shaft includes a wire lumen extending longitudinally through the shaft; and a wire positioned within the wire lumen and extending to the distal end of the shaft. A distal portion of the wire extends (i) out of a rear-side opening of the shaft and (ii) into a tip-side opening of the shaft such that an exposed portion of the wire is outside of the shaft between the rear-side opening and the tip-side opening. A distal end of the wire is coupled to the shaft; and the rear-side opening is circumferentially offset from the tip-side opening.

In other aspects, the medical device may include one or more of the following features. The tip-side opening is at least partially formed by a first pair of longitudinally-extending side walls, each side wall of the first pair of side walls extends from the wire lumen to a radially-outermost surface of the shaft, and each side wall of the first pair of side walls is substantially parallel to each other. The rear-side opening is at least partially formed by a second pair of longitudinally-extending side walls, each side wall of the second pair of side walls extends from the wire lumen to a radially-outermost surface of the shaft, each side wall of the second pair of side walls is substantially parallel to each other, and each side wall of the second pair of side walls is at least one of: (i) substantially perpendicular to the first pair of side walls, or (ii) are angled approximately forty-five degrees relative to the first pair of side walls. The rear-side opening is at least partially formed by a second pair of longitudinally-extending side walls, each side wall of the second pair of side walls extends from the wire lumen to a radially-outermost surface of the shaft, each side wall of the second pair of side walls is substantially longitudinally aligned with each other, and each side wall of the second pair of side walls is transverse to the first pair of side walls.

In other aspects, a medical device may comprise a shaft extending distally to a distal end, wherein the shaft includes a wire lumen extending longitudinally through the shaft; and a wire positioned within the wire lumen and extending to the distal end of the shaft; wherein a distal portion of the wire extends (i) out of a rear-side opening of the shaft and (ii) into a tip-side opening of the shaft such that an exposed portion of the wire is outside of the shaft between the rear-side opening and the tip-side opening; wherein a distal end of the wire is coupled to the shaft; and wherein the distal portion of the wire is configured to transition from (i) a first position in which the distal end of the shaft is substantially straight to (ii) a second position in which the distal end of the shaft is curved and the distal portion of the wire forms an S-shaped curve.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a perspective view of an exemplary medical system, including an insertion device and a medical device, according to aspects of this disclosure.

FIG. 2 illustrates a distal portion of a medical device, according to aspects of this disclosure.

FIG. 3 illustrates a side vice of a distal portion of a medical device, according to aspects of this disclosure.

FIGS. 4 and 5 illustrate front views of cross-sections of a medical device, according to aspects of this disclosure.

FIG. 6 illustrates a chart of various configurations of a distal portion of a medical device, according to aspects of this disclosure.

FIGS. 7A and 7B illustrate different side views of a distal portion of a medical device, according to aspects of this disclosure.

FIGS. 8A and 8B illustrate different side views of a distal portion of a medical device, according to aspects of this disclosure.

FIGS. 9A and 9B illustrate different side views of a distal portion of a medical device, according to aspects of this disclosure.

FIGS. 10A and 10B illustrate different side views of a distal portion of a medical device, according to aspects of this disclosure.

FIGS. 11A and 11B illustrate different side views of a distal portion of a medical device, according to aspects of this disclosure.

FIGS. 12A and 12B illustrate different side views of a distal portion of a medical device, according to aspects of this disclosure.

### DETAILED DESCRIPTION

The terms “proximal” and “distal” are used herein to refer to the relative positions of the components of an exemplary medical system and exemplary medical devices. When used herein, “proximal” refers to a position relatively closer to the exterior of the body or closer to a medical professional using the medical system or medical device. In contrast, “distal” refers to a position relatively further away from the medical professional using the medical system or medical device, or closer to the interior of the body. As used herein, the terms “comprises,” “comprising,” “having,” “including,” or other variations thereof, are intended to cover a non-exclusive inclusion, such that a system, device, or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value. The term “transverse” may mean angled relative to each other, not aligned, and is not limited to an arrangement perpendicular relative to each other. The term “angled” may mean having a non-zero degree angle relative to each other.

Examples of this disclosure include systems, devices, and methods for facilitating and/or improving the efficacy, efficiency, and/or safety of a medical procedure. Embodiments of the disclosure may relate to systems, devices, and methods for performing various medical procedures and/or treating portions of the bile duct, large intestine (colon), small intestine, cecum, esophagus, stomach, any other portion of the gastrointestinal tract, and/or any other suitable patient anatomy. Various embodiments described herein include single-use or disposable medical devices. Some aspects of the disclosure may be used in performing an endoscopic, arthroscopic, bronchoscopic, ureteroscopic, colonoscopic, or other type of procedure. For example, the disclosed aspects may be used with endoscopes, duodenoscopes, bronchoscopes, ureteroscopes, colonoscopes, catheters, diagnostic or therapeutic tools or devices, or other types of medical devices. One or more of the elements discussed herein could be metallic, plastic, or include a shape memory metal (such as nitinol), a shape memory polymer, a polymer, or any combination of biocompatible materials.

Reference will now be made in detail to examples of the disclosure described above and illustrated in the accompanying drawings. Wherever possible, the same reference numbers will be used throughout the drawings to refer to the same or like parts. It is noted that one or more aspects of the medical systems or devices discussed herein may be combined and/or used with one or more aspects of other medical systems or devices discussed herein.

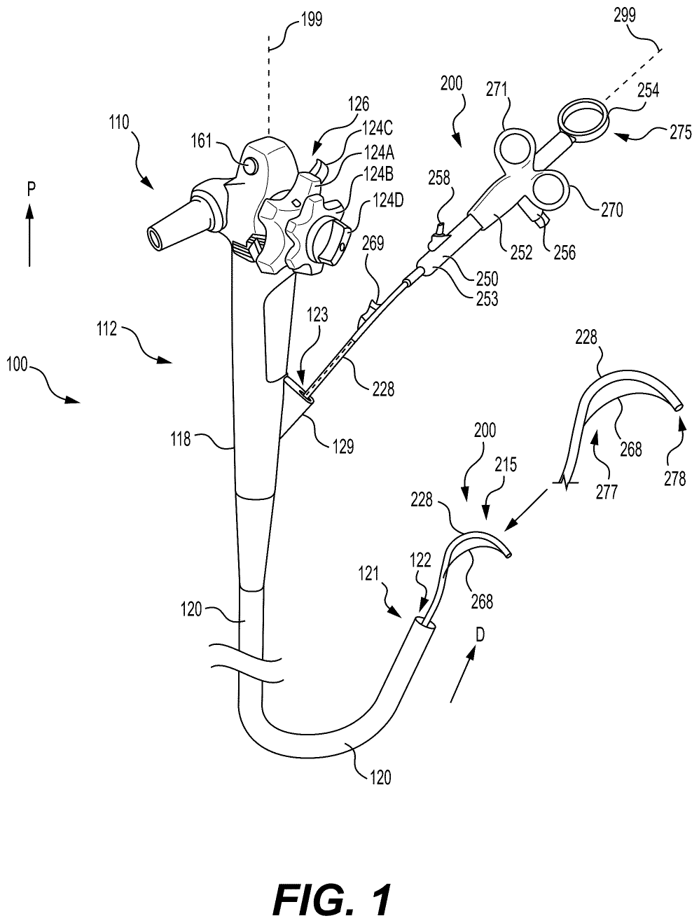

FIG. 1 illustrates a perspective view of an exemplary medical system 100 that includes an insertion device 112 and a medical device 200. Insertion device 112 includes an insertion device handle 118, including a handle body, and an insertion device shaft 120, for example, extending from a distal end of handle 118 to a distal end 121. Insertion device 112 also includes at least one working channel (not shown), for example, extending from insertion device handle 118 to a distal opening 122 in distal end 121. Insertion device 112 also includes a port 123, for example, in handle 118, and port 123 may connect to the working channel. Insertion device 112 may include a control device 126, for example, on a portion of handle 118, and one or more portions of control device 126 may be manipulated (e.g., rotated) to control a deflection of a portion of medical device shaft 120, for example, distal end 121.

Insertion device 112 may include a conduit 110. For example, insertion device handle 118 may be coupled to conduit 110. Conduit 110 may connect insertion device handle 118 to an external power source, processing software, one or more displays, one or more memory or storage devices, etc., for example, via an umbilicus (not shown). In this aspect, insertion device 112 may include one or more illumination devices and/or cameras at distal end 121, which may be powered and/or connected to processing software, one or more displays, a memory, etc. via one or more communication wires (not shown) within insertion device 112 and via conduit 110. Additionally, conduit 110 may connect insertion device handle 118 to one or more fluid sources, for example, an air source, a water source, etc. Conduit 110 may also connect insertion device handle 118 to a suction source. In these aspects, one or more valves coupled to or received within one or more apertures (not shown) in insertion device handle 118 may control the delivery of air or water and/or the application of suction through insertion device 112 to the area distal to distal end 121 of insertion device shaft 120.

Insertion device 112 may be a duodenoscope, an endoscope, a colonoscope, an ureteroscope, a bronchoscope, etc., or any other like device having a handle and a shaft. Insertion device 112 may have a central longitudinal axis 199. As mentioned, insertion device 112 may include control device 126, for example, on a proximal portion of handle 118. Control device 126 may be movable (e.g., rotatable) relative to handle 118, and may control the movement of a portion (e.g., distal end 121) of insertion device shaft 120. Control device 126 may include one or more dials or knobs. As shown in FIG. 1, control device 126 may include first and second knobs 124A and 124B, for example, each rotatable to deflect distal end 121 along two different planes. Control device 126 may include one or more locking mechanisms. As shown in FIG. 1, control device 24 may include two locking mechanisms 124C and 124D, for example, each engageable with one of knobs 124A and 124B to lock and/or unlock the position of the knob, and thus lock and/or unlock the position of distal end 121 of insertion device shaft 120. Alternatively, control device 126 may actuate or move one or more elevators in insertion device shaft 120, or otherwise actuate a cable driven function of insertion device 112. One or more buttons 161 may also be part of control device 126, and the one or more buttons may control a camera, a fluid injector, a light (e.g. a light emitting diode (LED) light), or any other feature of insertion device 112.

Medical device 200 may include a medical device handle 250 and a medical device shaft 228 extending from medical device handle 250 to a distal end 215. Medical device handle 250 may include a movable handle portion or an actuator portion 252 and a main handle body 253. Actuator portion 252 may move in the proximal or distal direction relative to main handle body 253, and may include two ring portions 270, 271 configured to receive one or more of a user's fingers or thumbs. Main handle body 253 may also include a ring portion 254 at a proximalmost end 275 of main handle body 253, and ring portion 254 may be configured to receive one or more of a user's fingers or thumbs. As discussed in detail below, movement of actuator portion 252 relative to main handle body 253 may control the movement of one or more cutting wires 268 and control a deflection of distal end 215 of medical device shaft 228.

Main handle body 253 may include a first port 258, and first port 258 may be fluidically connected to a first lumen extending longitudinally through a portion of main handle body 253, through shaft 228, to a distal end 278 of shaft 228. First port 258 may be configured to receive fluid, such as contrast fluid. A second port 256 may be positioned on actuator portion 252, and second port 256 may be configured to receive electrical energy to electrify a portion of medical device 200, such as the one or more cutting wires 268. For example, a user may connect a source of electrical energy to second port 256, and second port 256 may be electrically connected to the one or more cutting wires 268 to transfer the electrical energy from the electrical energy source to the one or more cutting wires 268. Second port 256 may be configured to receive electrical energy similar to an electrical plug or socket. A third port 269 may be positioned at a proximal portion of shaft 228, and may be connected to a second lumen extending longitudinally through shaft 228 to distal end 278 of shaft 228. Third port 269 may be configured to receive a guidewire. Although third port 269 is shown at a proximal portion of shaft 228, third port 269 may be positioned on main handle body 253.

Medical device shaft 228 may be delivered through port 129 of handle 118, and through a working channel to extend from distal end 121 of shaft 120 of insertion device 112. In these aspects, distal end 215 of medical device shaft 228 may be extended from distal end 121 of insertion device 112, and may be controlled via one or more of control device 126, movement of medical device handle 250 relative to insertion device handle 118, and movement of actuator portion 252 relative to main handle body 253.

As mentioned, medical device 200 includes medical device handle 250 and medical device shaft 228 extending from medical device handle 250 to distal end 215. Medical device handle 250 includes main handle body 253 and actuator portion 252, with actuator portion 252 being movable relative to main handle body 253. As discussed below, one or more cutting wires 268 may be connected to actuator portion 252, and the one or more cutting wires 268 may be coupled to a distal portion of medical device shaft 228. Movement of actuator portion 252 relative to main handle body 253 may control the movement of the one or more cutting wires 268, and also control the deflection of the distal end 215 of medical device 200. Additionally, although not shown, medical device handle 250 may include one or more frictional and/or locking elements to help control and/or lock the relative movement of actuator portion 252 relative to main handle body 253.

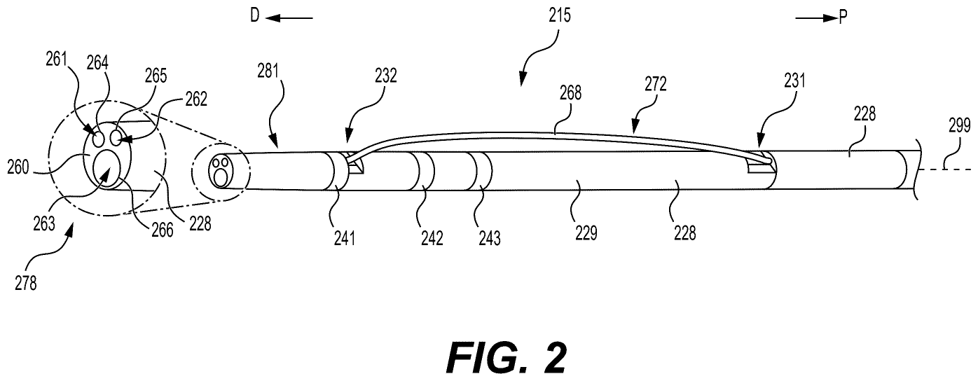

Distal end 215 of medical device 200 may include an end effector, for example, an energy delivery portion or an end effector 277. End effector 277 may be the one or more cutting wires 268 or a portion of the one or more cutting wires 268, and may be electrically connected to second port 256. As will be discussed further herein below with relation to FIG. 2, a portion of the one or more cutting wires 268 that is exposed outside of shaft 228 at distal end 215 may be moved via movement of actuator portion 252 relative to main handle body 253, which may help the user to deliver energy and/or cut tissue and/or treat tissue.

FIG. 2 illustrates a magnified view of distal end 215 of medical device 200, with distal end 278 shown enlarged in the left portion (facing the page) of FIG. 2. One or more cutting wires 268 is shown as a single cutting wire 268 in FIG. 2. Cutting wire 268 may extend through a lumen 265 of shaft 228, may extend out of a first opening 231 in shaft 228, and may extend into a second opening 232 in shaft 228. A distal portion 272 of cutting wire 268 may be positioned outside of shaft 228, and a proximal portion of cutting wire 268 (not shown) may be positioned entirely within shaft 228 (e.g., within lumen 265). A distal end of cutting wire 268 may be positioned within lumen 265, and, in some examples, may be fixed to an interior portion of lumen 265. Distal portion 272 of cutting wire 268 may extend radially outward, relative to a central longitudinal axis 299 of shaft 228, from a radially-outermost surface 229 of shaft 228. Cutting wire 268 may be moveable proximally or distally through lumen 265, such as via actuation of actuator portion 252 of handle 250.

Openings 231, 232 may be longitudinally aligned with each other (as shown in FIG. 2) and may extend from radially-outermost surface 229 of shaft 228 to lumen 265. In some examples, each opening 231, 232 may be square shaped, rectangular shaped, circular, or any other shape. Shaft 228 may include one or more marker regions 241, 242, 243, which may be cylindrical colored bands or colored regions around shaft 228. Marker regions 241, 242, 243 may be configured to facilitate identifying the orientation of shaft 228 during an operation, and may be spaced from each other to designate a specific reference distance for a user to refer to during an operation.

As shown in FIG. 2, distal end 278 may include an opening 262 to lumen 265 configured to receive the cutting wire 268, an opening 261 to lumen 264 configured to receive fluid (e.g. contract fluid), and opening 263 to lumen 266 configured to receive a guidewire. Each of openings 261, 262, 263 may be positioned on a distal front face 260 of distal end 278. In some examples, lumens 264, 265 may have a smaller diameter than lumen 266. Shaft 228 may include a tapered distal portion 281 extending distally to distal front face 260, which may facilitate maneuvering shaft 228 into body lumens during a procedure.

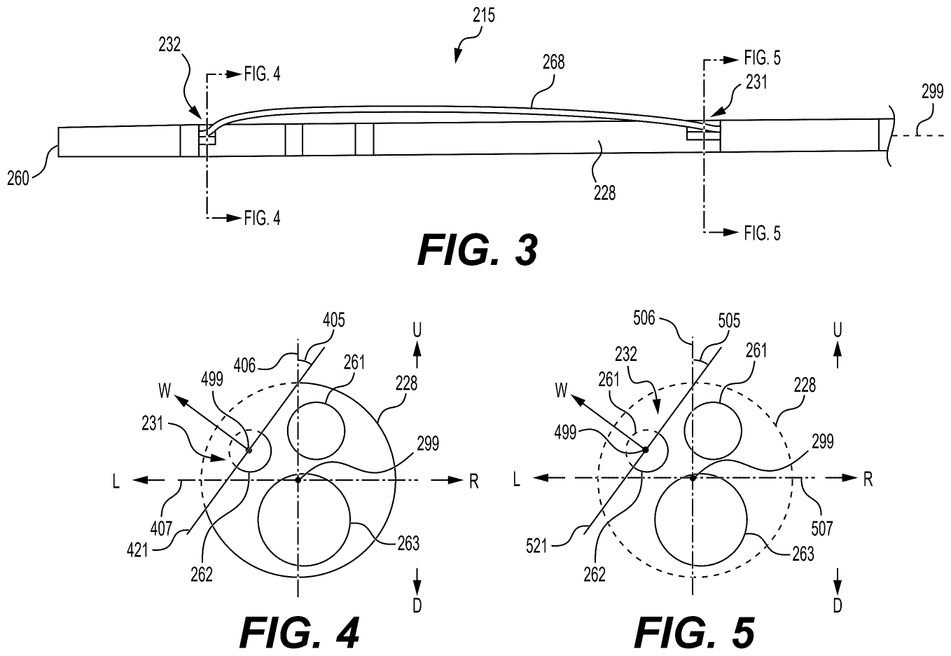

FIG. 3 illustrates a side view of distal end 215, including cutting wire 268, openings 231, 232, shaft 228, and distal front face 260. A dotted-lined arrow across opening 232 designates where along shaft 228 the cross-section shown in FIG. 4 is taken, and a dotted-lined arrow across opening 231 designates where along shaft 228 the cross-section shown in FIG. 5 is taken.

FIG. 4 illustrates a cross-sectional view of shaft 228 taken through opening 232, which may be referred to as the tip-side opening 232. A first line 406 is shown extending through central longitudinal axis 299 (shown extending into the page) of shaft 228, and is shown as a reference for the up and down directions (also arrows U an D in FIG. 4 are shown as reference to the up and down directions, respectively.) A second line 407 is shown extending through central longitudinal axis 299 of shaft 228, and is shown as a reference for the left and right directions (also arrows L and R in FIG. 4 are shown as reference to the left and right directions, respectively.) Each of lines 406 and 407 may be approximately perpendicular to central longitudinal axis 299 and to one another. A reference line 421 is shown extending across the radially-outer surface of shaft 228 where opening 232 begins, which extends through a central longitudinal axis 499 of lumen 265. A reference angle 405 is shown between reference line 421 and first line 406, which may be approximately forty-five degrees, thirty degrees, or any other suitable angle. Cutting wire 268 may extend out of opening 231 in a direction substantially perpendicular to reference line 421, and the direction that cutting wire 268 extends is shown by arrow W in FIG. 4.

FIG. 5 illustrates a cross-sectional view of shaft 228 taken through opening 231, which may be referred to as the rear-side opening 231. A first line 506 is shown extending through central longitudinal axis 299 (shown extending into the page) of shaft 228, and is shown as a reference for the up and down directions (also arrows U an D in FIG. 5 are shown as reference to the up and down directions, respectively.) Each of lines 506 and 507 may be approximately perpendicular to central longitudinal axis 299 and to one another. A second line 507 is shown extending through central longitudinal axis 299 of shaft 228, and is shown as a reference for the left and right directions (also arrows R an L in FIG. 5 are shown as reference to the right and left directions, respectively.) A reference line 521 is shown extending across the radially-outer surface of shaft 228 where opening 231 begins, which extends through the central longitudinal axis 499 of lumen 265. A reference angle 505 is shown between reference line 521 and first line 506, which may be approximately forty-five degrees, thirty degrees, or any other suitable angle. Cutting wire 268 may extend out of opening 231 in a direction substantially perpendicular to reference line 521, and the direction that cutting wire 268 extends is shown by arrow W in FIG. 5.

As will be discussed in further detail herein below, the orientation of openings 231, 232 formed in shaft 228 may cause cutting wire 268 to extend outward from shaft 228 at different directions (e.g. different directions of arrow W). By specifically configuring the orientation, shape, spacing, and sizing of openings 231, 232, the direction cutting wire 268 extends from shaft 228 may be adjusted to facilitate specific desirable angles of attack (or cutting angles) for particular procedures.

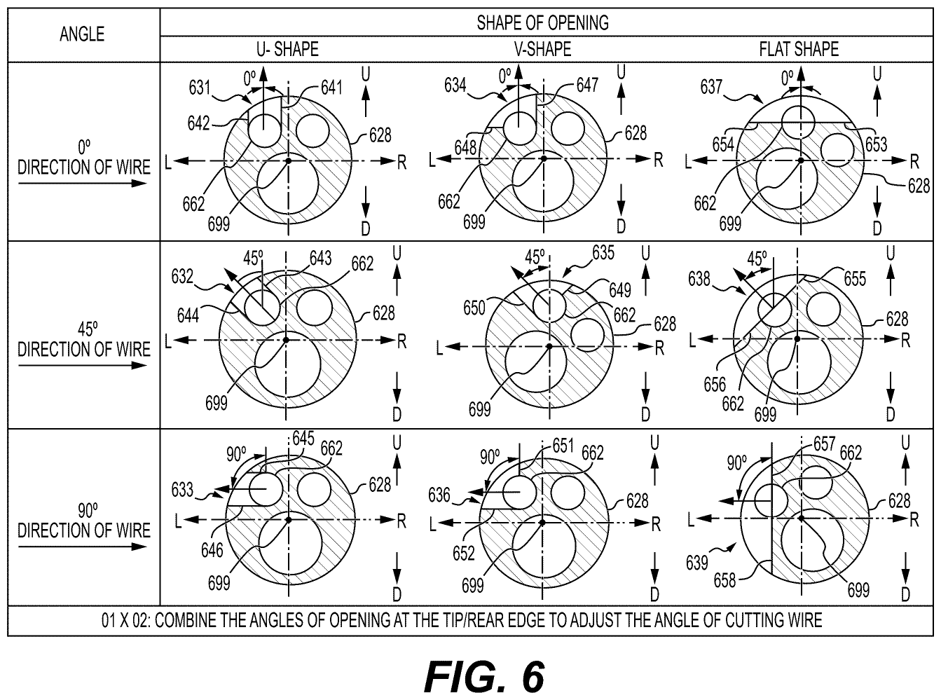

FIG. 6 illustrates a chart of various openings 631-639 formed in a shaft, such as shaft 228, to connect a lumen with outside of the shaft. The top of the chart in FIG. 6 lists three different types of shapes of openings: U-shaped openings 631-633, V-shaped openings 634-636, and flat shaped openings 637-639. The left side of the chart in FIG. 6 lists three different angles associated with the direction that the cutting wire 268 extends out of each opening 631-639: 0 degrees, 45 degrees, and 90 degrees. Each of these angles, as described hereinabove with relation to FIGS. 4 and 5, may represent the angle at which the cutting wire 268 exits lumen 662, for example relative to the up and down directions for that particular shaft.

For example, opening 631 has a 0 degree reference angle because cutting wire 268 extends upwards out of opening 631 in a direction parallel to the up direction (shown as the U arrow in FIG. 6.) Note up and down directions (shown as U and D arrows throughout FIG. 6) may be different for different embodiments of shaft 628, as shown in FIG. 6. The up and down directions may be defined relative to handle 250, a control unit connected to shaft 628, or any other portion of medical device 200 fixed to shaft 628. For example, up and down directions may be defined relative to first port 258 of handle 250, with the up direction extending approximately ninety degrees offset from first port 258 and the down direction extending approximately ninety degrees offset from the first port 258 and one hundred and eighty degrees offset from the up direction (e.g. rotationally offset relative to a central longitudinal axis of shaft 628). As shown in FIG. 6 purely for illustrative purposes, the shaft 628 shown with a U-shaped opening 631 and a zero degree direction of cutting wire 268 (i.e. shown in the top left of the chart in FIG. 6) may have up and down directions defined differently compared to the up and down directions defined for the shaft 628 shown with a flat shaped opening 637 and a zero degree direction of cutting wire 268 (i.e. shown in the top right of the chart in FIG. 6). The up and down directions may be different depending on the orientation of shaft 628 relative to handle 250, which is not consistent across the chart of shafts 628 shown in FIG. 6.

Opening 631 is formed by two side walls 641, 642 extending from lumen 662 to a radially-outermost surface of a shaft 628. Each side wall 641, 642 may be planar and may be substantially parallel to each other. Although not shown in FIG. 6, any of the openings 631-639 may also be formed by a distal side wall at a distal end of each opening 631-639 and a proximal side wall at a proximal end of each opening 631-639, and any of these distal side walls and/or proximal side walls may be planar or curved. Reference axes are shown throughout FIG. 6 extending through a central longitudinal axis 699 of shaft 628, in both the up-down direction and the left-right direction.

As shown in FIG. 6, U-shaped opening 632 directs cutting wire 268 outward at a 45 degree angle relative to the up direction (shown as the U arrow in FIG. 6). Opening 632 is formed by two side walls 643, 644 extending from lumen 662 to a radially-outermost surface of shaft 628. Each side wall 643, 644 may be planar and may be substantially parallel to each other. Each side wall 643, 644 may extend from diametrically opposed points on the outer circumference of lumen 662. Sidewalls 643, 644 may be rotated forty-five degrees relative to sidewalls 641, 642. U-shaped opening 633 directs cutting wire 268 outward at a 90 degree angle relative to the up direction (shown as the U arrow in FIG. 6). Opening 633 is formed by two side walls 645, 646 extending from lumen 662 to a radially-outermost surface of shaft 628. Each side wall 643, 644 may be planar and may be substantially parallel to each other. Sidewalls 645, 646 may be rotated ninety degrees relative to sidewalls 641, 642.

V-shaped opening 634 directs cutting wire 268 outward at a 0 degree angle relative to the up direction (shown as the U arrow in FIG. 6), or substantially parallel to the up direction. Opening 634 is formed by two side walls 647, 648 extending from lumen 662 to a radially-outermost surface of shaft 628. Each side wall 647, 648 may be planar, and side wall 647 may be substantially perpendicular to side wall 648. Sidewall 647 may be substantially parallel to the up-down direction, and sidewall 648 may be substantially perpendicular to the up direction and substantially parallel to left-right direction.

V-shaped opening 635 directs cutting wire 268 outward at a 45 degree angle relative to the up direction (shown as the U arrow in FIG. 6). Opening 635 is formed by two side walls 649, 650 extending from lumen 662 to a radially-outermost surface of shaft 628. Each side wall 649, 650 may be planar, and side wall 649 may be substantially perpendicular to side wall 650. V-shaped opening 636 directs cutting wire 268 outward at a 90 degree angle relative to the up direction (shown as the U arrow in FIG. 6), or substantially perpendicular to the up direction. Note the up and down directions for shaft 628 with opening 635 are different compared to the up and down directions for shaft 628 with opening 634. Side wall 649 may extend along a plane extending through the diameter of lumen 662, and sidewall 650 may extend tangentially from a curved surface of shaft 628 forming lumen 662.

Opening 636 is formed by two side walls 651, 652 extending from lumen 662 to a radially-outermost surface of shaft 628. Each side wall 651, 652 may be planar, and side wall 651 may be substantially perpendicular to side wall 652.

Flat shaped opening 637 directs cutting wire 268 outward at a 0 degree angle relative to the up direction (shown as the U arrow in FIG. 6), or substantially parallel to the up direction. Opening 637 is formed by two side walls 653, 654 extending from lumen 662 to a radially-outermost surface of shaft 628. Each side wall 653, 654 may be planar, and side wall 653 may be substantially parallel to side wall 654. Side wall 653 may extend outward from lumen 662 in a first direction, and side wall 654 may extend outward from lumen 662 in a second direction opposite from the first direction. Flat shaped opening 638 directs cutting wire 268 outward at a 45 degree angle relative to the up direction (shown as the U arrow in FIG. 6). Sidewalls 653, 654 may extend along surfaces of the side walls of shaft 628, and each of side walls 653, 654 may form a radially-outermost surface of shaft 628 relative to central longitudinal axis 699.

Opening 638 is formed by two side walls 655, 656 extending from lumen 662 to a radially-outermost surface of shaft 628. Each side wall 655, 656 may be planar, and side wall 655 may be substantially parallel to side wall 656. Side wall 655 may extend outward from lumen 662 in a first direction, and side wall 656 may extend outward from lumen 662 in a second direction opposite from the first direction. Flat shaped opening 639 directs cutting wire 268 outward at a 90 degree angle relative to the up direction (shown as the U arrow in FIG. 6), or substantially perpendicular to the up direction. Opening 639 is formed by two side walls 657, 658 extending from lumen 662 to a radially-outermost surface of shaft 628. Each side wall 657, 658 may be planar, and side wall 657 may be substantially parallel to side wall 658. Side wall 657 may extend outward from lumen 662 in a first direction, and side wall 658 may extend outward from lumen 662 in a second direction opposite from the first direction. Note shafts 628 shown with openings 637, 638, 639 may be coupled to handle 250 at different positions (e.g. rotated relative to longitudinal axis 699), and thus the up and down directions are defined differently for each of these shafts with openings 637, 638, 639.

Any of side walls 641-658 may extend the entire longitudinal length of an opening 631-639, and may be any suitable length. Any of openings 631-639 may be incorporated into medical devices of this disclosure, for example to replace one or more of openings 231, 232. By utilizing a combination of opening shapes, such as a combination of the exemplary openings 631-639 shown in FIG. 6, for the tip-side opening (e.g. opening 232) and the rear-side opening (e.g. opening 231) in medical device 200, cutting-wire may be configured to extend outward from shaft 228 at a predetermined angle and curvature.

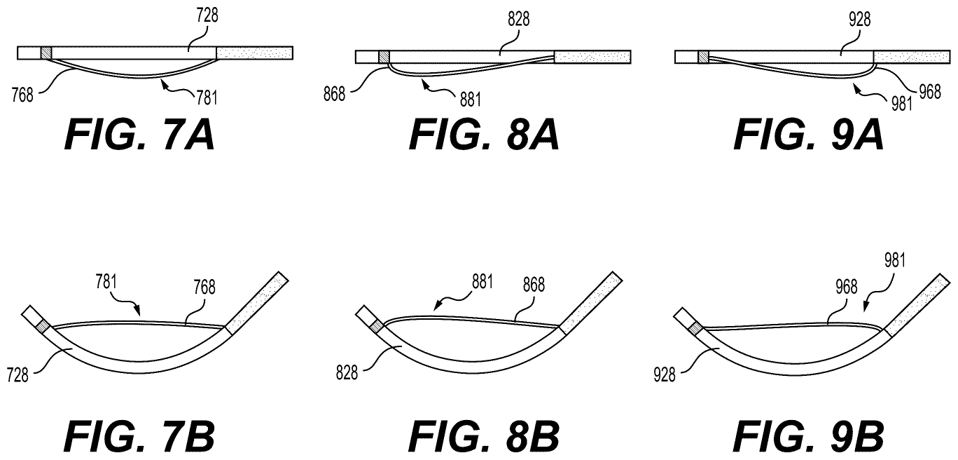

FIGS. 7A-12B illustrate exemplary configurations of the tip-side opening (e.g. opening 232) and the rear-side opening (e.g. opening 231) in medical device 200, to illustrate the curvature and direction of a cutting wire with different tip-side opening and rear-side opening combinations. All of the distal sections shown in FIGS. 7A-12B are in an actuated state, with actuator portion 252 of handle 250 either (i) pushed distally to move the cutting wire out of a rear-side opening of shaft, or (ii) pulled proximally to bend a distal portion of the shaft and pull a distal end of the cutting wire and the tip-side opening proximally.

FIGS. 7A and 7B illustrate side views of an exemplary shaft 728 and cutting wire 768 when both tip-side opening (e.g. opening 232) and the rear-side opening (e.g. opening 231) direct cutting wire 768 outward at a 90 degree angle relative to the up direction. Alternatively, both tip-side opening and the rear-side opening may direct cutting wire 768 outward at a 45 degree angle relative to the up direction. In shaft 728 shown in FIGS. 7A and 7B, both tip-side opening and the rear-side opening may be any one of openings 632, 633, 635, 636, 638, 639. When actuated (e.g. by pulling actuator portion 252 of handle 250), cutting wire 768 may move away from shaft 728. In an actuated state (shown in FIGS. 7A and 7B), the exposed portion of cutting wire 768 outside of shaft 728 may be curved and may include a peak portion 781, or an apex, approximately equidistant from each of the tip-side opening and the rear-side opening. Peak portion 781 may be the portion of cutting wire 768 that is the farthest from shaft 728 in an actuated state.

FIGS. 8A and 8B illustrate side views of an exemplary shaft 828 and cutting wire 868 when tip-side opening (e.g. opening 232) directs cutting wire 868 outward at a 45 degree angle relative to the up direction, and rear-side opening (e.g. opening 231) direct cutting wire 868 outward at a 0 degree angle relative to the up direction. Alternatively, tip-side opening may direct cutting wire 868 outward at a 90 degree angle relative to the up direction, and rear-side opening may direct cutting wire 868 outward at a 0 degree angle or a 45 degree angle relative to the up direction. In shaft 828 shown in FIGS. 8A and 8B, tip-side opening may be any one of openings 632, 635, 638; and the rear-side opening may be any one of openings 631, 634, 637. When actuated (e.g. by pulling actuator portion 252 of handle 250), cutting wire 868 may move away from shaft 828. In an actuated state (shown in FIGS. 8A and 8B), the exposed portion of cutting wire 868 outside of shaft 828 may be curved and may include a peak portion 881, or an apex, closer to the tip-side opening than the rear-side opening.

FIGS. 9A and 9B illustrate side views of an exemplary shaft 928 and cutting wire 968 when tip-side opening (e.g. opening 232) directs cutting wire 968 outward at a 0 degree angle relative to the up direction, and rear-side opening (e.g. opening 231) direct cutting wire 968 outward at a 90 degree angle relative to the up direction. Alternatively, the tip-side opening may direct cutting wire 968 outward at a 45 degree angle relative to the up direction, and rear-side opening may direct cutting wire 968 outward at a 0 degree or a 45 degree angle relative to the up direction. In shaft 928 shown in FIGS. 9A and 9B, tip-side opening may be any one of openings 631, 634, 637; and the rear-side opening may be any one of openings 633, 636, 639. When actuated (e.g. by pulling actuator portion 252 of handle 250), cutting wire 968 may move away from shaft 928. In an actuated state (shown in FIGS. 9A and 9B), the exposed portion of cutting wire 968 outside of shaft 928 may be curved and may include a peak portion 981, or an apex, closer to the rear-side opening than the tip-side opening.

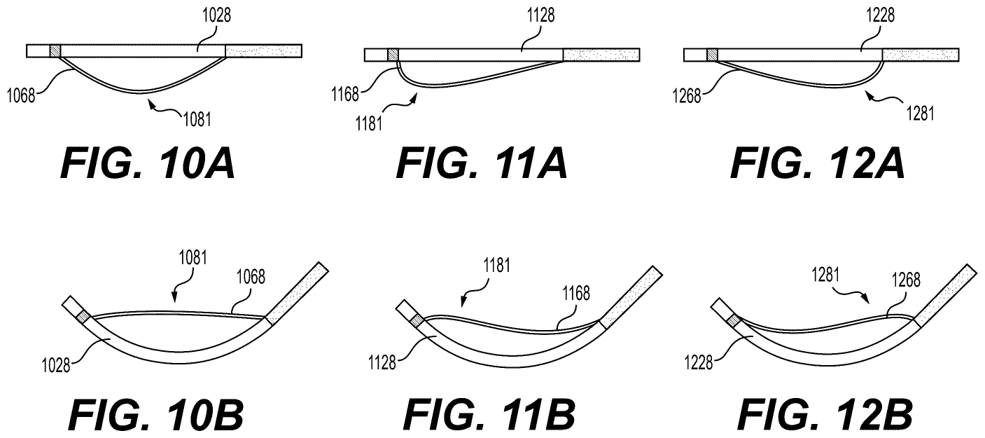

FIGS. 10A and 10B illustrate side views of an exemplary shaft 1028 and cutting wire 1068 when both tip-side opening (e.g. opening 232) and the rear-side opening (e.g. opening 231) direct cutting wire 1068 outward at a 45 degree angle relative to the up direction. Alternatively, rear-side opening may direct cutting wire 1068 outward at a 90 degree angle relative to the up direction. In shaft 1028 shown in FIGS. 10A and 10B, both tip-side opening and the rear-side opening may be any one of openings 632, 635, 638. When actuated (e.g. by pushing actuator portion 252 of handle 250), cutting wire 1068 may move away from shaft 1028. In an actuated state (shown in FIGS. 10A and 10B), the exposed portion of cutting wire 1068 outside of shaft 1028 may be curved and may include a peak portion 1081, or an apex, approximately equidistant from each of the tip-side opening and the rear-side opening. Peak portion 1081 may be the portion of cutting wire 1068 that is the farthest from shaft 1028 in an actuated state.

FIGS. 11A and 11B illustrate side views of an exemplary shaft 1128 and cutting wire 1168 when tip-side opening (e.g. opening 232) directs cutting wire 1168 outward at a 90 degree angle relative to the up direction, and rear-side opening (e.g. opening 231) directs cutting wire 1168 outward at a 0 degree angle relative to the up direction. Alternatively, the tip-side opening directs cutting wire 1168 outward at a 45 degree angle relative to the up direction, and the rear-side opening directs cutting wire 1168 outward at a 0 degree angle relative to the up direction; or the tip-side opening directs cutting wire 1168 outward at a 90 degree angle relative to the up direction, and the rear-side opening directs cutting wire 1168 outward at a 45 degree angle relative to the up direction In shaft 1128 shown in FIGS. 11A and 11B, tip-side opening may be any one of openings 633, 636, 639; and the rear-side opening may be any one of openings 631, 634, 637. When actuated (e.g. by pushing actuator portion 252 of handle 250), cutting wire 1168 may move away from shaft 1128. In an actuated state (shown in FIGS. 11A and 11B), the exposed portion of cutting wire 1168 outside of shaft 1128 may be curved and may include a peak portion 1181, or an apex, closer to the tip-side opening than the rear-side opening. The exposed portion of cutting wire 1168 may include both convex and concave portions relative to shaft 1128.

FIGS. 12A and 12B illustrate side views of an exemplary shaft 1228 and cutting wire 1268 when tip-side opening (e.g. opening 232) directs cutting wire 1268 outward at a 0 degree angle relative to the up direction, and rear-side opening (e.g. opening 231) directs cutting wire 1268 outward at a 45 degree angle relative to the up direction. Alternatively, the tip-side opening directs cutting wire 1268 outward at a 0 degree angle relative to the up direction, and rear-side opening directs cutting wire 1268 outward at a 90 degree angle relative to the up direction; or the tip-side opening directs cutting wire 1268 outward at a 0 degree angle relative to the up direction, and rear-side opening directs cutting wire 1268 outward at a 0 or a 45 degree angle relative to the up direction. In shaft 1228 shown in FIGS. 12A and 12B, tip-side opening may be any one of openings 631, 634, 637; and the rear-side opening may be any one of openings 632, 635, 638. When actuated (e.g. by pushing actuator portion 252 of handle 250), cutting wire 1268 may move away from shaft 1228. In an actuated state (shown in FIGS. 12A and 12B), the exposed portion of cutting wire 1268 outside of shaft 1228 may be curved and may include a peak portion 1281, or an apex, closer to the rear-side opening than the tip-side opening. Peak portion 1281 may be the portion of cutting wire 1268 that is the farthest from shaft 1228 in an actuated state. The exposed portion of cutting wire 1268 may include both convex and concave portions relative to shaft 1228.

By adjusting the direction and style of openings 231, 232, 631-639 for each of the tip-side opening and the rear-side opening in medical device 200, the direction of movement and the actuated shape of the one or more cutting wires 268 may be adjusted to optimize the use of medical device 200 and facilitate an appropriate cutting angle for a given procedure. As shown in FIGS. 7A-12B, the shape of the exposed portion of the cutting wire (e.g. the portion used to cut tissue), when in an actuated state, may vary depending on the type and position of openings 231, 232.

Any of the configurations of rear-side openings and tip-side openings shown in FIGS. 7A-12B, and any other combinations of rear-side and tip-side openings, may be incorporated into medical device 200. Any of cutting wires 768, 868, 968, 1068, 1168, 1268; shafts 728, 828, 928, 1028, 1128, 1228; and openings 631-639 may be incorporated into medical device 200, for example to replace shaft 228, one or more cutting wires 268, and/or openings 231, 232.

In an endoscopic sphincterotomy, a spincterotome may be positioned into the bile duct transpapillarily and to incise the papillary bile duct including the common channel with radiofrequency current (e.g. by deploying cutting wire 268 and applying electrical energy to cutting wire 268). Endoscopic sphincterotomy is a procedure essential for various biliopancreatic endoscopic therapies and diagnoses, including release of papillary stenosis and stent placement, and precut is performed for the patients with difficulty in bile duct intubation. When the long axis of the papilla is set in the direction of 12 o'clock relative to the distal end 215 of the medical device 200 (e.g. a distal end of a sphincterotome), it is considered safe to incise the papilla in the direction from 11 to 12 o'clock. Regarding hemorrhage, the distribution of blood vessels in the direction from 10 to 11 o'clock is less than other areas of the papilla, and it is described that the incision in this direction has low risk of hemorrhage. Considering the bile duct as a central axis or center of a clock as a reference, moreover, there is also an opinion that the papillary bile duct runs to the direction of 11 to 12 o'clock up to near the hooding fold, and then gradually returns to the direction of 12 o'clock. Therefore, an incision in the direction from 11 to 12 o'clock is safe and recommended to reduce the risk of hemorrhage and perforation due to endoscopic sphincterotomy. By configuring the angle of attack of cutting wire 268 in medical device 200, a user may more easily achieve an incision direction from 11 to 12 o'clock when distal end 215 is positioned in the papilla.

Various aspects discussed herein may allow for an insertion device (e.g. insertion device 112) and a medical device (e.g., medical device 200) to be delivered to a treatment site, for example, to perform endoscopic sphincterotomy or otherwise treat the treatment site. A user may move the distal end 121 of insertion device 112 to position distal end 215 of medical device 200 at the treatment site. Actuation of actuator portion 252 on handle 250 may move cutting wire 268 towards the treatment site at one or more angles, which may help the user to perform the treatment at the treatment site. Specifically, the angle of attack of cutting wire 268 towards treatment site may help the user achieve a more optimal incision to treat the patient.

Accordingly, various aspects discussed herein may help to improve the efficacy of treatment and/or recovery from a procedure, for example, a procedure to treat a treatment side. Various aspects discussed herein may help to reduce and/or minimize the duration of the procedure, and/or may help reduce risks of inadvertent contact with non-target tissue or other material during delivery, repositioning, or removal of a medical system for the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device comprising
a handle including an actuator;
a shaft extending from the handle, wherein the shaft includes a wire lumen extending longitudinally through the shaft; and
a wire positioned within the wire lumen and extending from the handle to a distal end of the shaft;
wherein a distal portion of the wire extends (i) out of a rear-side opening of the shaft and (ii) into a tip-side opening of the shaft such that an exposed portion of the wire is outside of the shaft between the rear-side opening and the tip-side opening; and
wherein a distal end of the wire is coupled to the shaft.

2. The medical device of claim 1, wherein the rear-side opening is longitudinally aligned with the tip-side opening.

3. The medical device of claim 1, wherein the rear-side opening is at least partially formed by a first pair of longitudinally-extending side walls, wherein each side wall of the first pair of side walls extends from the wire lumen to a radially-outermost surface of the shaft, and wherein each side wall of the first pair of side walls are substantially parallel to each other.

4. The medical device of claim 3, wherein the tip-side opening is at least partially formed by a second pair of longitudinally-extending side walls, wherein each side wall of the second pair of side walls extends from the wire lumen to a radially-outermost surface of the shaft, wherein each side wall of the second pair of side walls are substantially parallel to each other, and wherein the second pair of side walls are transverse from the first pair of side walls.

5. The medical device of claim 3, wherein the tip-side opening is at least partially formed by a second pair of longitudinally-extending side walls, wherein each side wall of the second pair of side walls extends from the wire lumen to a radially-outermost surface of the shaft, wherein each side wall of the second pair of side walls is substantially parallel to each other, and wherein the second pair of side walls are at least one of: (i) substantially perpendicular to the first pair of side walls, or (ii) are angled approximately forty-five degrees relative to the first pair of side walls.

6. The medical device of claim 1, wherein the tip-side opening is at least partially formed by a first pair of longitudinally-extending side walls, wherein each side wall of the first pair of side walls extends from the wire lumen to a radially-outermost surface of the shaft, and wherein each side wall of the first pair of side walls is substantially longitudinally aligned with each other.

7. The medical device of claim 6, wherein the rear-side opening is at least partially formed by a second pair of longitudinally-extending side walls, wherein each side wall of the second pair of side walls extends from the wire lumen to a radially-outermost surface of the shaft, wherein each side wall of the second pair of side walls is substantially longitudinally aligned with each other, and wherein the second pair of side walls are transverse to the first pair of side walls.

8. The medical device of claim 1, wherein the rear-side opening is at least partially formed by a first pair of longitudinally-extending side walls, wherein each side wall of the first pair of side walls extends from the wire lumen to a radially-outermost surface of the shaft, and wherein a first side wall of the first pair of side walls extends substantially perpendicular to a second side wall of the first pair of side walls.

9. The medical device of claim 8, wherein the tip-side opening is at least partially formed by a second pair of longitudinally-extending side walls, wherein each side wall of the second pair of side walls extends from the wire lumen to a radially-outermost surface of the shaft, wherein a third side wall of the second pair of side walls extends substantially perpendicular to a fourth side wall of the second pair of side walls, wherein the first side wall is angled relative to the third side wall and the second side wall is angled relative to the fourth side wall.

10. The medical device of claim 1, wherein the tip-side opening is circumferentially offset from the rear-side opening.

11. The medical device of claim 1, wherein the wire is configured to receive electrical energy.

12. The medical device of claim 1, wherein the shaft further includes a second lumen configured to receive a guidewire.

13. The medical device of claim 9, wherein the shaft further includes a third lumen configured to receive fluid.

14. The medical device of claim 10, wherein the wire, when in an actuated configuration, is configured to include both a convex portion and a concave portion relative to the shaft.

15. The medical device of claim 1, wherein the wire, when in an actuated configuration, is configured to include a peak portion, and wherein the peak portion is closer to the tip-side opening than the rear-side opening when the wire is in an actuated state.

16. A medical device comprising
a shaft extending distally to a distal end, wherein the shaft includes a wire lumen extending longitudinally through the shaft; and
a wire positioned within the wire lumen and extending to the distal end of the shaft;
wherein a distal portion of the wire extends (i) out of a rear-side opening of the shaft and (ii) into a tip-side opening of the shaft such that an exposed portion of the wire is outside of the shaft between the rear-side opening and the tip-side opening;
wherein a distal end of the wire is coupled to the shaft; and
wherein the rear-side opening is circumferentially offset from the tip-side opening.

17. The medical device of claim 16, wherein the tip-side opening is at least partially formed by a first pair of longitudinally-extending side walls, wherein each side wall of the first pair of side walls extends from the wire lumen to a radially-outermost surface of the shaft, and wherein each side wall of the first pair of side walls is substantially parallel to each other.

18. The medical device of claim 17, wherein the rear-side opening is at least partially formed by a second pair of longitudinally-extending side walls, wherein each side wall of the second pair of side walls extends from the wire lumen to a radially-outermost surface of the shaft, wherein each side wall of the second pair of side walls is substantially parallel to each other, and wherein each side wall of the second pair of side walls is at least one of: (i) substantially perpendicular to the first pair of side walls, or (ii) are angled approximately forty-five degrees relative to the first pair of side walls.

19. The medical device of claim 17, wherein the rear-side opening is at least partially formed by a second pair of longitudinally-extending side walls, wherein each side wall of the second pair of side walls extends from the wire lumen to a radially-outermost surface of the shaft, wherein each side wall of the second pair of side walls is substantially longitudinally aligned with each other, and wherein each side wall of the second pair of side walls is transverse to the first pair of side walls.

20. A medical device comprising:
a shaft extending distally to a distal end, wherein the shaft includes a wire lumen extending longitudinally through the shaft; and
a wire positioned within the wire lumen and extending to the distal end of the shaft;
wherein a distal portion of the wire extends (i) out of a rear-side opening of the shaft and (ii) into a tip-side opening of the shaft such that an exposed portion of the wire is outside of the shaft between the rear-side opening and the tip-side opening;
wherein a distal end of the wire is coupled to the shaft; and
wherein the distal portion of the wire is configured to transition from (i) a first position in which the distal end of the shaft is substantially straight to (ii) a second position in which the distal end of the shaft is curved and the distal portion of the wire forms an S-shaped curve.
